(19) United States
(12) Patent Application Publication
(10) Pub. No.: US 2005/0226432 A1
(43) Pub. Date: Oct. 13, 2005

(54) Method for automatically reducing noises in an earphone

(76) Inventor: Kun-Ta Lee, Taipei (TW)

Correspondence Address: LEE, KUN-TA, 235 P.O. BOX 10-69, Chung-Ho, Taipei, TW

(21) Appl. No.: 10/813,295

(22) Filed: Mar. 31, 2004

(51) Int. Cl. A61F 11/06; G10K 11/16; H03B 29/00

(52) U.S. Cl. 381/71.6; 381/72; 381/74

(57) ABSTRACT

A method for automatically reducing noises in an earphone using an audio transceiver is disclosed. The audio transceiver includes an audio transmitter, an audio auto-adjusting module, an audio amplifying module, an audio detecting and receiving module, a power set and a manual adjusting button. The method comprises the steps of: receiving external interference audio signals in the audio detecting and receiving module of the audio transceiver; generating mixing frequency signals in the audio auto-adjusting module; the mixing frequency signals having frequencies near the frequencies of the external interference audio signal; a phase difference of about 180 degree from the interference audio signal and amplitudes approximately equal to those of the external interference audio signals; transferring the mixing frequency signals to the audio amplifying module for amplifying the mixing frequency signals; and transmitting the amplified mixing frequency signals so as to cancel the external interference audio signals.

### FIELD OF THE INVENTION

The present invention relates to a method for automatically reducing noises in an earphone, which generates mixing frequency signals with same frequencies, opposite phases and same amplitudes of external interference audio signals so as to cancel the external interference audio signals.

### BACKGROUND OF THE INVENTION

In general, sounds unaccepted by people are called as noises which are measured with an unit of decibel (briefly dB). Sounds are classified as pure sound and compound sound. Noises are un-periodic compound sounds. If one lives in a noisy environment, the person will feel uneasy.

In general, sources of noises are classified as static noises which are generated from, for example, plants, business areas, amusement fields, fixed amplifiers, building fields, etc. Another kind of sound is called as dynamic noises which are generated from, for example, vehicles, airplanes, movable amplifiers, etc. Those in noisy environment for longer time period will deteriorate faculty of memory, have anxiety and sleeplessness so as to get diseases about the heart or blood vessels. Thereby, it is very important to reduce noises.

With reference to FIG. 1, a prior art ear mask 1 of noise-proof is illustrated. In that, a head ring 11 is arranged to enclose two sides of a frame 10. An inner side of the head ring 11 has mask bodies 12 which are adhered on the ears of users so as to isolate external sounds. However, the mask bodies 12 cannot effectively isolate external interference audio signals.

### SUMMARY OF THE INVENTION

Accordingly, the primary object of the present invention is to provide a method for automatically reducing noises in an earphone, which generates mixing frequency signals with same frequencies, opposite phases and same amplitudes of external interference audio signals so as to cancel the external interference audio signals.

To achieve above objects, the present invention provides a method for automatically reducing noises in an earphone using an audio transceiver. The audio transceiver includes an audio transmitter, an audio auto-adjusting module, an audio amplifying module, an audio detecting and receiving module, a power set and a manual adjusting button. The method comprises the steps of: receiving external interference audio signals in the audio detecting and receiving module of the audio transceiver; generating mixing frequency signals in the audio auto-adjusting module; the mixing frequency signals having frequencies near the frequencies of the external interference audio signal; a phase difference of about 180 degrees from the interference audio signal and amplitudes equal to those of the external interference audio signals; transferring the mixing frequency signals to the audio amplifying module for amplifying the mixing frequency signals; and transmitting the amplified mixing frequency signals so as to cancel the external interference audio signals.

The various objects and advantages of the present invention will be more readily understood from the following detailed description, when read in conjunction with the appended drawing.

### BRIEF DESCRIPTION OF THE DRAWINGS

FIG. 1 is a perspective view of a prior art earphone.

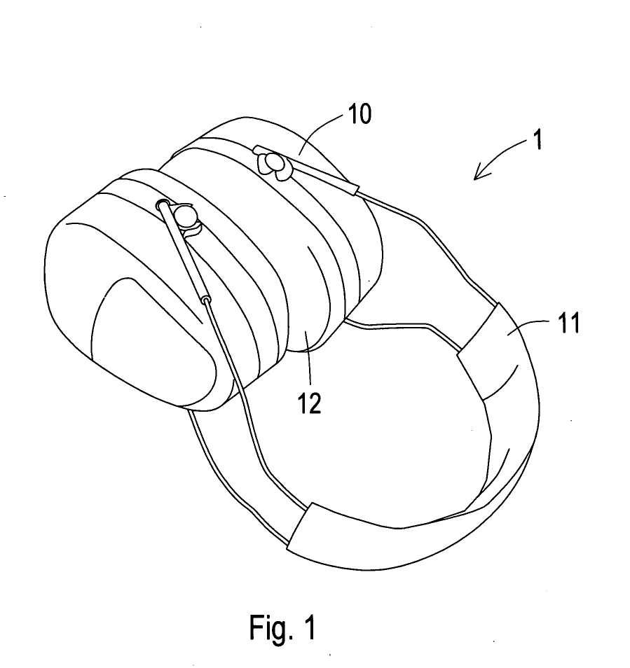

FIG. 2 is a schematic block view about the audio signal processing of the present invention.

FIG. 3 shows the relation of the audio transceiver of the present invention.

FIG. 4 shows the waveforms of the mixing frequency signals and interference audio signals according to the present invention.

FIG. 5 shows the installation of the audio transceiver according to the present invention.

FIG. 6 shows one embodiment of the present invention.

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

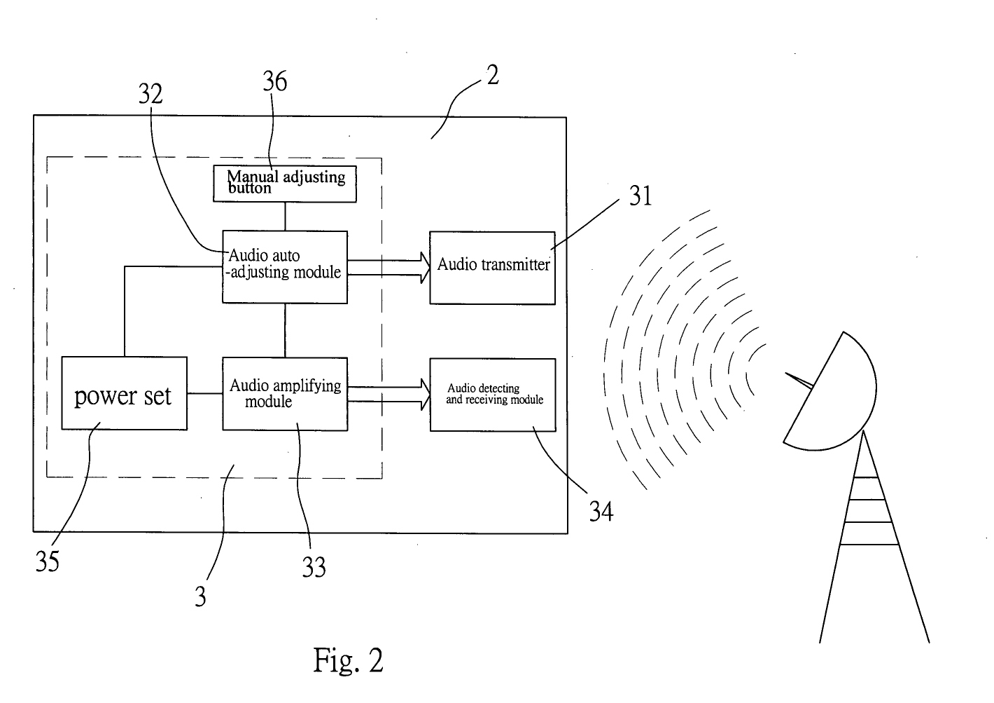

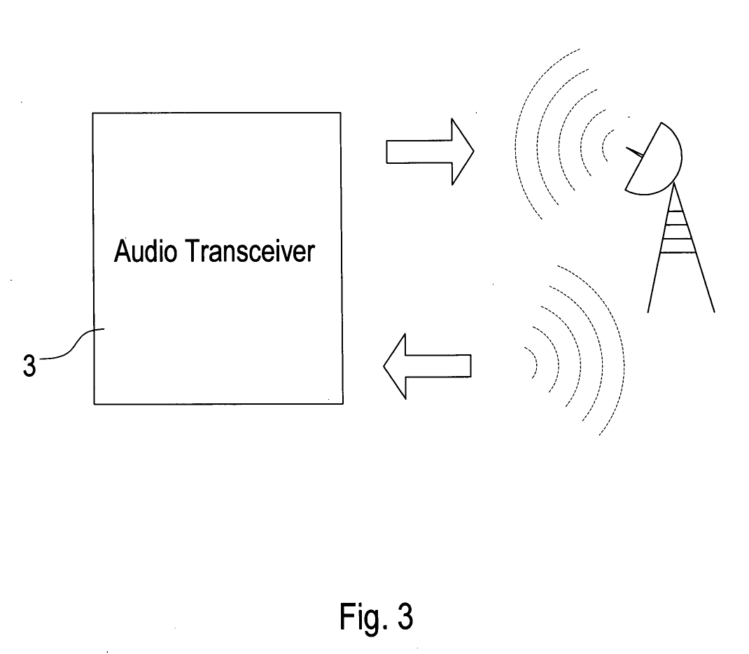

Referring to FIGS. 2 and 3, a method for reducing noises in an earphone of the present invention is illustrated. The method is used to an earphone system of an audio transceiver, in that an audio transceiver 3 is installed in an earphone 2. The audio transceiver 3 includes an audio transmitter 31, an audio auto-adjusting module 32, an audio amplifying module 33, an audio detecting and receiving module 34, a power set 35, and a manual adjusting button 36. The audio detecting and receiving module 34 serves to receive at least one frequency band of a mixing frequency signal. The mixing frequency signal passes through the audio auto-adjusting module 32 for generating a corresponding audio signal to the audio amplifying module 33. Then audio signal is transmitted by the audio transmitter 31 for transmitting a balance sound wave to cancel noise and thus external interferences are reduced.

The present invention serves to generate a balance mechanism by canceling sound wave so as to solve the problem of the unbalance of the right and left ears due the nerves control of the human brain. One side of the earphone 2 is installed with a manual adjusting button 36 for adjusting the transmitting sound wave in the left or right ear according to the sound decibel (dB) value. The transmitting sound wave is adjusted to have amplitudes equal to those of noises so as to cancel the noises.

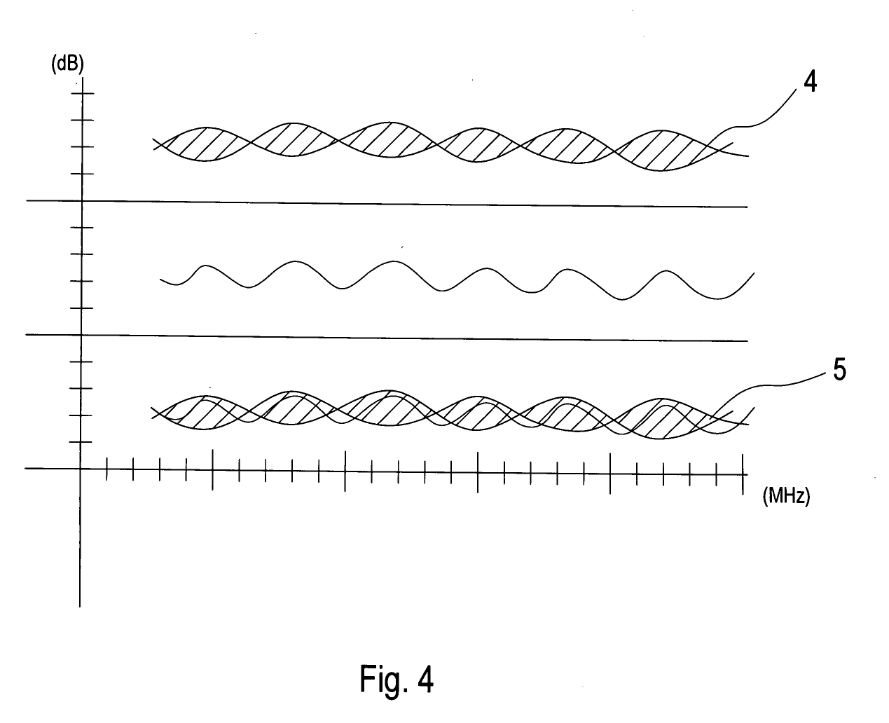

Referring to FIG. 4, an embodiment of the present invention will be described herein. In the audio transceiver 3 of the present invention, a mixing frequency 5 is generated according to the external interference audio signal 4 so that the mixing frequency 5 can cancel the interference audio signal 4. Thereby, the user wearing the earphone will feel comfortable. Thus, when external interference audio signal interferes the audio transceiver 3, the audio transceiver 3 generates mixing frequency to cancel the interference so as to reduce noise.

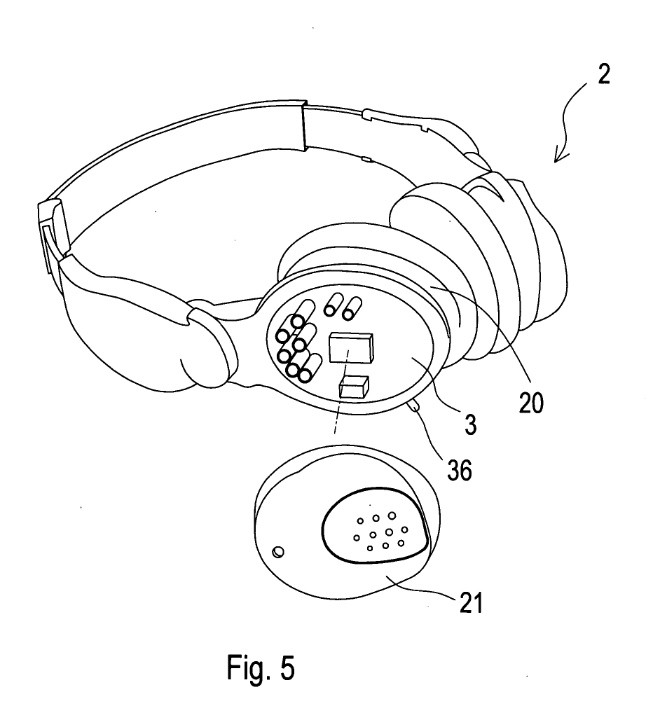

With reference to FIG. 5, the audio transceiver 3 of the present invention is installed inn mask 20 of an earphone 2. A cover 21 covers the mask 20 for protecting the audio transceiver 3.

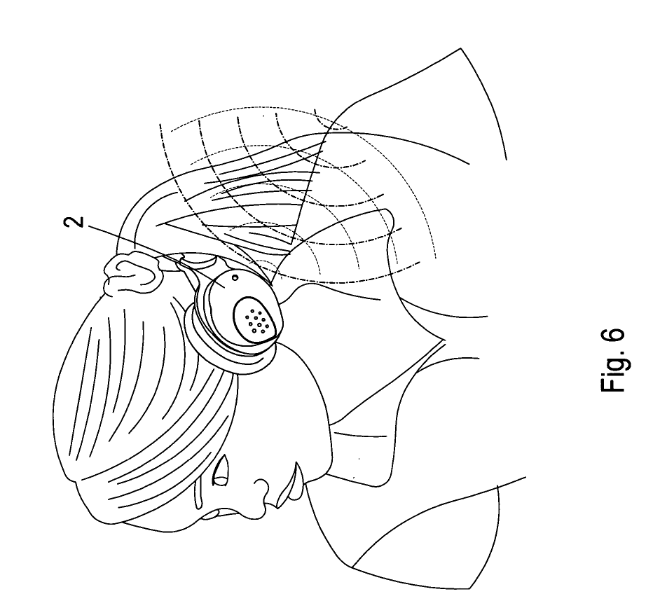

With reference to FIG. 6, when the user wears the earphone 2 of the present invention, the audio transmitter 31 generates a mixing frequency with a phase difference of 180 degrees to external interference audio signal and having an amplitude same as that of the external interference audio signal so as to cancel the external interference audio signal 4. Thus noise can be reduced.

Advantages of the present invention will be described here. The main advantage of the present invention is to reduce external interference audio signals so as to buffer the impact of sound pressure and thus users wearing the earphone will feel easy and comfortable. Thereby, the problem of the unbalance of sound pressures within ears can be solved. Thereby, the user can concentrate the attention to work and thus working efficiency is increased. Moreover, the present invention provides a function of adjusting the unbalance in the left and right ears. Moreover, the present invention is suitable in various places, such as bus stations, super markets, driving cars, planes, boats, offices, etc.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for automatically reducing noises in an earphone using an audio transceiver; the audio transceiver including an audio transmitter, an audio auto-adjusting module, an audio amplifying module, an audio detecting and receiving module, a power set and an manual adjusting button; the method comprising the steps of:
receiving external interference audio signals in the audio detecting and receiving module of the audio transceiver;
generating mixing frequency signals in the audio auto-adjusting module; the mixing frequency signals having frequencies near the frequencies of the external interference audio signal; a phase difference of about 180 degree from the interference audio signal and amplitudes approximately equal to those of the external interference audio signals;
transferring the mixing frequency signals to the audio amplifying module for amplifying the mixing frequency signals; and
transmitting the amplified mixing frequency signals by the audio transmitter so as to cancel the external interference audio signals.

2. The method for automatically reducing noises in an earphone as claimed in claim 1, wherein the manual adjusting button serves to adjust the mixing frequency signals emitted from the right or left ears so as to balance the sound pressures in the two ears of the user.

3. The method for automatically reducing noises in an earphone as claimed in claim 1, wherein the audio detecting and receiving module serves to receive external interference audio signals.

4. The method for automatically reducing noises in an earphone as claimed in claim 1, wherein the output signals from the audio transmitter has a frequency capable of canceling the external interference audio signals.

5. The method for automatically reducing noises in an earphone as claimed in claim 1, wherein the manual adjusting button is at one side of the earphone for adjusting the output signals from the audio transmitter so that the output signals serve to cancel the sound waves and reduce noises in the earphone.
